(19) United States
(12) Patent Application Publication
(10) Pub. No.: US 2020/0316353 A1
(43) Pub. Date: Oct. 8, 2020

(54) RADIATION DERMATITIS PREVENTION APPARATUS AND METHOD

(71) Applicants: Colette Spears, Charlotte, NC (US); Lenard Spears, Charlotte, NC (US)

(72) Inventors: Colette Spears, Charlotte, NC (US); Lenard Spears, Charlotte, NC (US)

(21) Appl. No.: 16/758,313

(22) PCT Filed: Oct. 29, 2018
(86) PCT No.: PCT/US2018/057987

§ 371 Date: Apr. 22, 2020

Related U.S. Application Data
(60) Provisional application No. 62/578,646, filed on Oct. 30, 2017.

(51) Int. Cl. A61M 35/00

(52) U.S. Cl. CPC A61M 35/10; A61M 2210/1007; A61M 2209/088

(57) ABSTRACT

A radiation dermatitis prevention apparatus includes: a pack (10), including: a pocket (12) including a face panel (16) and a back panel (18), wherein the panels (16, 18) are secured to each other along their respective peripheries in such a way as to define an opening (20), wherein the face panel (16) is constructed from a material suitable for contact with a patients skin and which readily permits fluid to flow through its thickness; and an insert (14) sized to fit in the opening (20) and made of a material which is suitable for holding and releasing a radiation cream in viscous fluid form.

### BACKGROUND OF THE INVENTION

This invention relates generally to radiation dermatitis, and specifically to its prevention during cancer radiotherapy.

Radiation dermatitis is one of the most common side effects of radiotherapy for cancer, affecting approximately 95% of patients receiving radiotherapy. Acute injury, which occurs within hours to weeks after radiation exposure, results from immediate structural tissue damage, generation of short-lived free radicals, irreversible double-stranded breaks in nuclear and mitochondrial DNA, and initiation of an inflammatory response in the epidermis and dermis. Repeated exposure to low-dose ionizing radiation does not allow time for cells to repair DNA or tissue damage. The accumulation of radiation-induced changes to dermal vasculature, appendageal structures, and epidermal stem cells result of the progression of radiation dermatitis through characteristic stages of severity.

Its onset varies depending on the radiation dose intensity in the normal tissue sensitivity of individuals. It is confined to areas of skin they have been irradiated, and the skin changes are sharply demarcated. Radiation-induced dermatitis can be classified as acute or chronic.

Acute radiation dermatitis occurs within 90 days of exposure to radiation the patient may have skin changes raising from faint erythema (reddening) and desquamation (peeling skin) to necrosis (death of skin cells) and ulceration, depending on the severity of the reaction.

The national Cancer institute (USA) has developed a four stage criteria for the classification of acute radiation dermatitis:

Grade 1—faint erythema or desquamation.

Grade 2—moderate to brisk erythema or patchy, moist desquamation combined to skin folds and creases. Moderate swelling.

Grade 3—confluence, moist desquamation greater than 1.5 cm diameter, which is not confined to the skin folds. Pitting space edema (severe swelling).

Grade 4—skin necrosis or ulceration of full thickness dermis (middle layer skin).

Onset of chronic radiation dermatitis may occur from 15 days to 10 years or more after the beginning of radiation therapy. It is an extension of the acute process involving further inflammatory changes in the skin.

Chronic radiation-induced changes in the skin are characterized by:

Disappearance of follicular structures (pores).

Increasing collagen and damaged elastic fibers in the dermis.

Fragile surface skin (epidermis).

Telangiectasia (prominent blood vessels).

Secondary skin cancers may also result from repeated exposure to radiation therapy, most commonly basal cell carcinoma.

Radiation dermatitis has a profound effect on the quality of a patient's life, due to pain and discomfort. In addition, it may be the cause of premature interruption of radiation therapy, resulting in inadequate disease treatment.

It is possible to treat radiation dermatitis by the application of a cream. One problem with the use of creams is that they are inconvenient and messy. Furthermore, the area of the affected skin needs to be protected from additional damage by UV exposure

### BRIEF SUMMARY OF THE INVENTION

This problem is addressed by a radiation dermatitis prevention system comprising a pack including a plurality of fabric layers which is configured to apply a treatment cream against a patient's skin.

According to one aspect of the technology described herein, a radiation dermatitis prevention apparatus includes: a pack, including: a pocket including a face panel and a back panel, wherein the panels are secured to each other along their respective peripheries in such a way as to define an opening, wherein the face panel is constructed from a material suitable for contact with a patient's skin and which readily permits fluid to flow through its thickness; and an insert sized to fit in the opening and made of a material which is suitable for holding and releasing a radiation cream in viscous fluid form.

### BRIEF DESCRIPTION OF THE DRAWINGS

The invention may be best understood by reference to the following description taken in conjunction with the accompanying drawing figures in which:

FIG. 1 is a schematic, exploded perspective view showing an exemplary radiation dermatitis prevention pack;

FIG. 2 is a schematic diagram showing a portion of the pack of FIG. 1 in contact with a patient's body part;

FIG. 3 is a schematic top plan view of a belt that may be used as part of a securing apparatus for a pack;

FIG. 4 is a schematic top plan view of a shoulder strap assembly that may be used as part of a securing apparatus for a pack;

FIG. 5 is a schematic front elevation diagram showing packs secured in place against a patient's body using a securing apparatus; and

FIG. 6 is a schematic front perspective diagram showing another arrangement of packs secured in place against a patient's body using a securing apparatus.

### DETAILED DESCRIPTION OF THE INVENTION

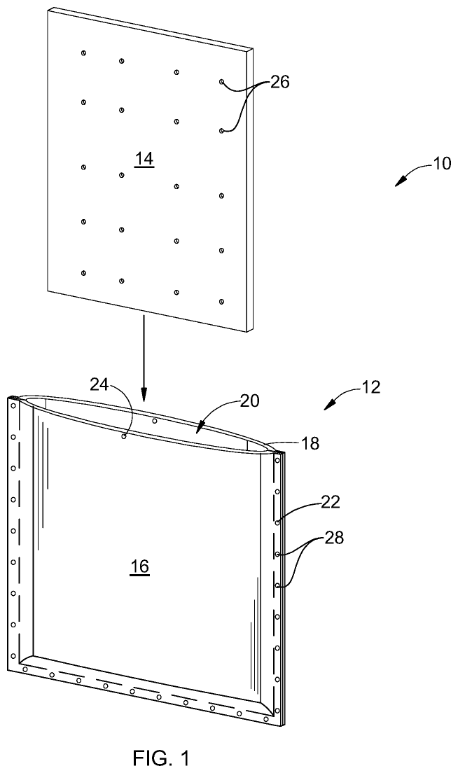

Referring to the drawings wherein identical reference numerals denote the same elements throughout the various views, FIG. 1 illustrates an exemplary radiation dermatitis prevention apparatus in the form of a pack 10 constructed according to an aspect of the present invention. The basic components of the pack 10 are a pocket 12 and an insert 14.

The pocket 12 comprises a face panel 16 and a back panel 18. The panels 16, 18 are secured to each other along their respective peripheries in such a way as to define an opening 20. In the illustrated example, the pocket 12 is depicted as having a rectangular shape, but the specific shape is not critical, and other shapes such as a polygon, circle, oval, or a curvilinear shape could be substituted. As will be described in more detail below, the pocket 12 may be shaped and sized to conveniently fit against different human body parts as required.

The face panel 16 is intended to contact the patient's skin and comprises a material which readily permits fluid to flow through its thickness. While any material could be used, the material should be non-toxic. For example, a non-toxic synthetic material could be used. Preferably, the material of the face panel 16 is a natural material (e.g. cotton, wool, hemp, etc.) rather than a synthetic, so as to avoid any chance of including toxic compositions. The material of the face panel 16 may be configured to be comfortable and avoid sticking to a patient's skin. The material of the face panel 16 may be configured to be comfortable and avoid sticking to a patient's skin. The material of the face panel 16 may be configured to be low-abrasion or non-abrasive. One nonlimiting example of a suitable material is a cotton mesh fabric.

The back panel 18 is intended to be exposed and comprises a material which is resistant to ultraviolet (UV) radiation in order to protect the patient's skin from UV radiation damage. Optionally, the back panel 18 may be constructed from a material which is moisture-proof in order to prevent the patient's clothing from getting stained by radiation cream when worn under clothing. The material of the back panel 18 may be a non-toxic synthetic material or a natural material (e.g. cotton, wool, hemp, etc.). One nonlimiting example of a suitable material is a knitted or woven wool fabric. If desired, a non-toxic chemical treatment or coating may be used with a natural material to provide UV resistance and/or moisture-proofing properties.

Any type of joint may be used between the face panel 16 and the back panel 18 so long as they are secured together, for example stitching, mechanical fasteners, folds, crimping, thermal bonding, or adhesives. In the illustrated example, conventional stitching 22 is used along three edges of the mutual perimeters of the face panel 16 and the back panel 18, leaving the fourth edge open to define the opening 20.

Optionally, a closure 24 (shown schematically) may be provided to selectively close off the opening 20 of the pocket 12. Non-limiting examples of suitable closures include buttons, snaps, hooks, turn button fasteners, clips, hook and loop fasteners, or zippers.

The insert 14 comprises a material which is suitable for holding and releasing a viscous fluid such as radiation cream. Optionally, the insert 14 may be constructed from a material which is moisture-proof in order to prevent the patient's clothing from getting stained by radiation cream when worn under clothing. For example, the insert 14 may comprise plastic sheeting. Optionally, the insert 14 may include surface apertures 26 to hold radiation cream, such as surface recesses, slots, grooves, or through-holes.

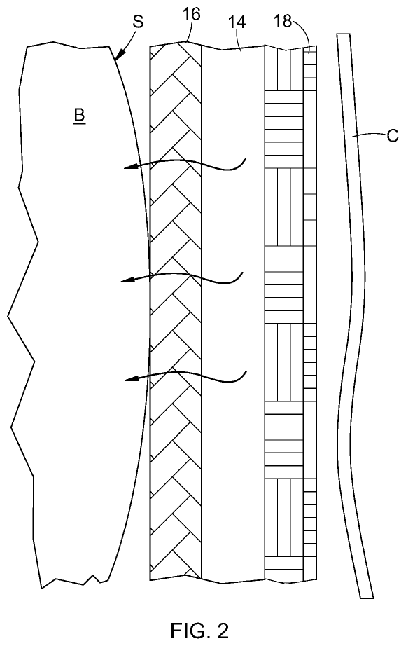

FIG. 2 illustrates how a patient may be protected by one or more of the packs 10 described above. The pack 10 is used (see FIG. 1) by first loading the insert 14 with radiation cream. The cream may be any composition which is safe for human body contact. The cream may be effective to reduce or prevent radiation dermatitis or symptoms thereof. Preferably the cream is a natural product rather than synthetic. One non-limiting example of a suitable cream is a mixture of shea butter and one or more essential oils.

Next, the insert 14 is placed into the pocket 12 and optionally secured by the closure 24. The pack 10 is then placed against a body part “B”, with the face panel 16 in contact with the skin “S”. The face panel 16 is configured so that it does not cause achieving or irritation to the skin S. Optionally, clothing “C” may overlie the pack 10. Cream is able to flow from the insert 14 through the face layer 16 to contact the skin S and provide a therapeutic benefit. Concurrently, the back layer 18 provides protection from further radiation damage by UV radiation.

The pack 10 may be placed against any body part as required. Typically, radiation dermatitis therapy involves placing the pack 10 against a body part and leaving it in place for a prescribed period of time. It is desirable that the patient be comfortable during this time and not have to hold the pack 10 in place. Also, it is desirable that the patient be able to undertake normal activities during the therapy.

Accordingly, the pack 10 may be configured for use with a securing apparatus, by providing it with one or more connectors arranged in an appropriate pattern. Nonlimiting examples of suitable connectors include buttons, snaps, hooks, turn button fasteners, clips, hook and loop fasteners, or zippers. In the example illustrated in FIG. 1, the pack 10 is provided with connectors 28 in the form of a plurality of spaced-apart snap fasteners arranged around its outer periphery. The snap fasteners or other connectors permit the pack 10 to be connected to another pack or to different kinds of securing apparatus.

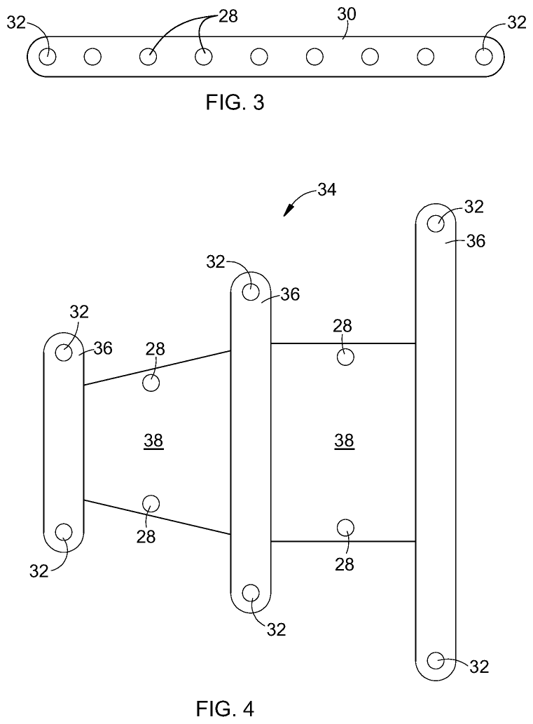

FIG. 3 illustrates an exemplary belt 30 constructed from an appropriate material such as leather, fabric, etc. and provided with end connectors 32. The belt 30 can be wrapped around a patient's torso or other body part and the end connectors 32 coupled to each other to secure the belt 30 in place. The belt 30 is provided with connectors 28 in the form of a plurality of spaced-apart snap fasteners arrayed along its length. The snap fasteners or other connectors permit the belt 30 to be connected to a pack 10 or to another piece of securing apparatus.

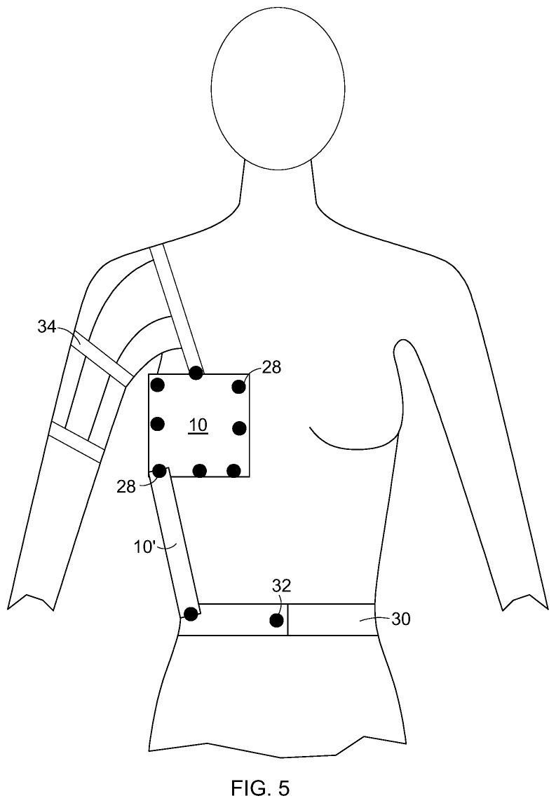

FIG. 4 illustrates an exemplary shoulder strap assembly 34 constructed from appropriate material such as leather, fabric, etc. The shoulder strap assembly 34 comprises two or more parallel spaced-apart straps 36 each having end connectors 28 at distal ends thereof, and a band of connecting material 38 interconnecting the straps 36. In addition to the end connectors 28, the shoulder strap assembly 34 may be provided with additional connectors 28 such as the illustrated snap fasteners. The end connectors 28 and/or the additional connectors 28 permit the shoulder strap assembly 34 to be connected to a pack 10 or to another piece of securing apparatus.

The securing apparatus such as the belt 30 and the shoulder strap assembly 34, or other similarly-configured components may be used to secure one or more of the packs 10 against one or multiple body parts for therapy.

FIG. 5 illustrates one particular example in which packs 10, 10′ are secured against a patient's breast and the side of the patient's thorax, respectively. In this example, the shoulder strap assembly 34 is placed over the patient's shoulder and the end connectors 32 are secured to the pack 10, suspending it in position over the patient's breast. Another pack 10′ contacting the patient's thorax is connected to the first pack 10 at its upper end, and its lower end is secured in position by a belt 30 as described above.

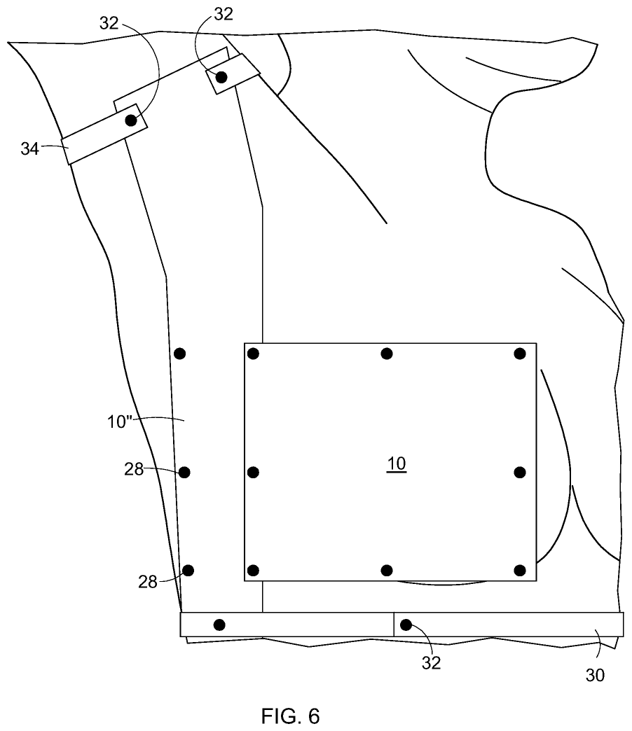

FIG. 6 illustrates another example in which a pack 10″ is secured against a patient's upper thorax and armpit. This would be used, for example, when the patient has had a lymph node removal in conjunction with breast surgery. This configuration may be used separately from or in addition to the configuration shown in FIG. 5. In this example, the shoulder strap assembly 34 (or another strap) is placed over the patient's shoulder and the end connectors 32 are secured to the pack 10″, suspending it in position against the patient's armpit. Its lower end is secured in position by a belt 30 as described above. Another pack 10 may be connected to the first pack 10″.

The pack and securing apparatus described herein has advantages over prior art methods for radiation dermatitis prevention. It provides an easy system and method for using treatment cream after radiation treatment. The system of packs is easily applied and can be customized to fit the necessary area affected by radiation treatment and surrounding traumatized body areas. The arrangement of connectors allows the packs to be connected and customized to achieve the necessary coverage needed. It provides the appropriate pressure for the pack to have contact with the skin in such a way that treatment effects are maximized.

The foregoing has described an apparatus and method for preventing radiation dermatitis. All of the features disclosed in this specification, and/or all of the steps of any method or process so disclosed, may be combined in any combination, except combinations where at least some of such features and/or steps are mutually exclusive. Each feature disclosed in this specification may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification, or to any novel one, or any novel combination, of the steps of any method or process so disclosed.

What is claimed is:

1. A radiation dermatitis prevention apparatus, comprising:
a pack (10), including:
a pocket (12) including a face panel (16) and a back panel (18), wherein the panels (16, 18) are secured to each other along their respective peripheries in such a way as to define an opening (20), wherein the face panel (16) is constructed from a material suitable for contact with a patient's skin and which readily permits fluid to flow through its thickness; and
an insert (14) sized to fit in the opening (20) and made of a material which is suitable for holding and releasing a radiation cream in viscous fluid form.

2. The apparatus of claim 1 wherein the face panel (16) is constructed from cotton mesh fabric.

3. The apparatus of claim 1 wherein the back panel (18) is constructed from a material which is resistant to ultraviolet radiation in order to protect a patient's skin from ultraviolet radiation damage.

4. The apparatus of claim 1 wherein the back panel (18) is constructed from a material which is moisture-proof in order to prevent a patient's clothing from being stained by radiation cream when worn under clothing.

5. The apparatus of claim 1, wherein the insert (14) is constructed from a material which is moisture-proof in order to prevent a patient's clothing from being stained by radiation cream when worn under clothing.

6. The apparatus of claim 5, wherein the insert (14) is constructed from plastic sheeting.

7. The apparatus of claim 1 wherein the insert (14) includes surface apertures (26) to hold radiation cream.

8. The apparatus of claim 1 wherein the pack (10) includes one or more connectors (28) arranged to permit the pack (10) to be connected to another pack (10′, 10″) or to a securing apparatus (30, 34).

9. The apparatus of claim 8 wherein the connectors (28) are snap fasteners.

10. The apparatus of claim 8 further comprising:
a belt (30) provided with end connectors (32) which can be coupled to each other to secure the belt (30) in place around a patient's waist; and
the belt (30) including at least one connector (28) along its length which permits the belt (30) to be connected to the pack (10) or to another piece of securing apparatus (30, 34).

11. The apparatus of claim 8, further comprising:
a shoulder strap assembly (34) including two or more parallel spaced-apart straps (36) each having end connectors (32) at distal ends thereof, and a band of connecting material (38) interconnecting the straps (36); and
additional connectors (28) which permit the shoulder strap assembly (34) to be connected to the pack (10) or to another piece of securing apparatus (30, 34).

12. A method of treating radiation dermatitis, comprising:
providing a pack (10), including:
a pocket (12) including a face panel (16) and a back panel (18), wherein the panels (16, 18) are secured to each other along their respective peripheries in such a way as to define an opening (20), wherein the face panel (16) is constructed from a material suitable for contact with a patient's skin and which readily permits fluid to flow through its thickness, and wherein the pocket (12) includes one or more connectors (28); and
an insert (14) sized to fit into the opening (20) and made of a material which is suitable for holding and releasing a radiation cream in viscous fluid form;
loading the insert (14) with radiation cream;
placing the insert (14) into the pocket (12); and
placing the pack (10) against a body part (B), with the face panel (16) in contact with the skin (S) of the body part (B), such that the radiation cream is able to flow from the insert (14) through the face layer (16) to contact the skin (S) and provide a therapeutic benefit.

13. The method of claim 12, further comprising:
providing a belt (30) provided with end connectors (32) which can be coupled to each other to secure the belt (30) in place around a patient's waist, the belt (30) including at least one connector (28) along its length; and
connecting the belt (30) to the pack (10) with their connectors mutually engaged, such that the belt holds the pack (10) against one or multiple body parts.

14. The method of claim 12, further comprising:
providing a shoulder strap assembly (34) including two or more parallel spaced-apart straps (36) each having end connectors (32) at distal ends thereof, and a band of connecting material (38) interconnecting the straps (36), and additional connectors (28); and
connecting the should strap assembly (34) to the pack (10) with their connectors mutually engaged, such that the belt holds the pack (10) against one or multiple body parts.

15. The method of claim 12, further comprising connecting two or more of the packs (10, 10′, 10″) together with their connectors mutually engaged.
